(19) United States
(12) Patent Application Publication
(10) Pub. No.: US 2009/0069313 A1
(43) Pub. Date: Mar. 12, 2009

(54) Novel MAO-A inhibitors

(75) Inventors: Debendranath Dey, Union City, CA (US); Gajendra Singh, Chennai (IN); Partha Neogi, Union City, CA (US); Uma Ramachandran, Chennai (IN); Chithra Santhanagopalan, Chennai (IN)

Correspondence Address: OLIFF & BERRIDGE, PLC, P.O. BOX 320850, ALEXANDRIA, VA, 22320-4850, US

(73) Assignee: ORCHID RESEARCH LABORATORIES LTD., CHENNAI (IN)

(21) Appl. No.: 11/896,902

(22) Filed: Sep. 6, 2007

(51) Int. Cl. A61K 31/5377; C07D 277/16; A61K 31/426; C07D 209/34; A61P 25/24; A61P 25/32; A61P 25/08; A61P 25/00; A61P 25/34; A61P 25/28; A61K 31/404; C07D 413/12; A61K 31/422

(52) U.S. Cl. 514/230.8; 548/183; 514/369; 548/229; 514/376; 544/144; 548/486; 514/418

(57) ABSTRACT

The present invention provides a new medical use for the compounds of the formula (I), their derivatives, analogs, tautomeric forms, stereoisomers, polymorphs, hydrates, solvates, pharmaceutically acceptable salts and compositions, metabolites and prodrugs thereof, as MAO-A inhibitors. Also included is a method for prophylaxis and treatment of mental disorders such as depression, in a mammal, comprising administering an effective amount of a compound of formula (I). These compounds are also effective in treating a disease, condition or disorder such as weight loss, obesity, bulimia, depression, bipolar disorders, psychoses, schizophrenia, behaviors, alcoholism, tobacco abuse, memory loss, Alzheimer's disease, dementia of aging, seizure disorders, epilepsy, attention deficit disorder, and Parkinson's disease, which is modulated by a cannabinoid receptor antagonist.

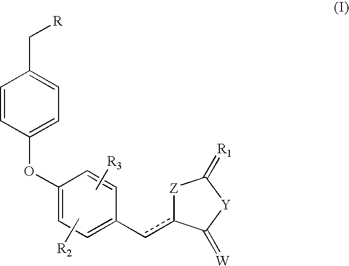

### FIELD OF THE INVENTION

A new use of and the method of treatment using a compound selected from a group consisting of the compound of formula (I), their derivatives, analogs, tautomeric forms, stereoisomers, polymorphs, hydrates, solvates, pharmaceutically acceptable salts and compositions, metabolites and prodrugs thereof.

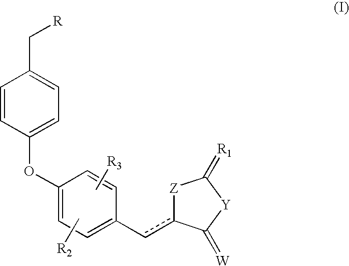

The new use of and method of treatment using the present MAO-A inhibitors, for prophylaxis and treatment of mental disorders such as depression.

### BACKGROUND OF THE INVENTION

This invention relates to new medical use for the compounds of formula (I) as MAO-A inhibitors. Monoamine oxidase (MAO) is the enzyme in the brain principally responsible for intraneuronal oxidation of biogenic amine neurotransmitters to inactive forms. It is understood to occur as two independent forms, normally designated MAO-A and MAO-B (White and Glassman, J. Neurochem., 28, 987-997, 1977 and Tipton et al, “Monoamine Oxidase and its Selective Inhibitors”, Beckmann and Riederer, Eds., Med. Probl. Pharmacopsychiat., 19 15-30, Karger, Basel, 1983). MAO inhibition has been found to elevate neurotransmitter concentrations in the brain.

MAO inhibitors are used therapeutically in the treatment of a wide variety of conditions, especially depression, particularly when characterized by anxiety, obsessional neuroses, or appetite disorders. However, a number of such compounds, for example isocarboxazid, phenelzine and tranylcypromine are non-selective, irreversible inhibitors of the enzyme and are characterized by an undesirable side effect associated with the ingestion of food or drink containing a high level of tyramine, for example, certain cheeses. When a patient receiving such a drug ingests such a product, then his blood pressure may be raised, sometimes to a dangerous level. Such patients are therefore instructed to avoid foods and beverages of this nature.

It is believed that the MAO inhibitors act to alleviate psychiatric disorders, such as depression, by increasing the concentration of one or more biogenic monoamines in the central nervous system. The monoamine oxidase enzyme plays an important role in the metabolic regulation of the monoamines since it catalyzes the biodegradation of the monoamines through oxidative deamination. By inhibiting MAO, the degradation of the monoamines is blocked, leading to an increase in the availability of the monoamines for their physiological functions. Among the physiologically active monoamines which are known substrates for MAO are: (a) the so-called “neurotransmitter” monoamines, such as the catecholamines (dopamine, epinephrine, norepinephrine) and the indoleamines (tryptamine, 5-hydroxytryptamine) (b) the so-called “trace” amines (o-tyramine, phenethylamine, tele-N-methylhistamine) and (c) tyramine.

The usefulness of the MAO inhibitors in treating depression has been limited because the administration of such agents can potentiate the pharmacological actions of certain food substances or drugs leading to dangerous and sometimes lethal effects. For example, persons receiving an MAO inhibitor must avoid the ingestion of foods which have a high tyramine content, such as cheese, because the MAO inhibitor will block the metabolic degradation of tyramine in the gut and liver resulting in high circulating levels of tyramine, with consequent release of catecholamines in the periphery, and finally serious hypertension. The potentiation by a MAO inhibitor of the pressor effect of tyramine arising from the ingestion of cheese and the hypertensive episode produced thereby, are commonly known as the “cheese reaction” or “cheese effect.” Moreover, persons on conventional MAO therapy cannot be given directly-acting sympathomimetic drugs or precursors thereof, which are themselves substrates for MAO (e.g. dopamine, epinephrine, norepinephrine or L-DOPA) and of indirectly-acting sympathomimetic drugs (amphetamines or over-the-counter cold, hay-fever, or weight control preparations which contain a vasoconstrictor). The potentiation of the pressor effects of indirectly-acting sympathomimetic drugs is especially profound. This is because such drugs act peripherally primarily by releasing catecholamines in nerve endings, resulting in dangerously elevated concentration of the liberated catecholamines if the metabolic degradation of the catecholamines via MAO is blocked. In addition, a particular MAO inhibitor should not be used in combination with another MAO inhibitor or with hypotensive agents, dibenzapine antidepressants, meperidine, CNS depressants and anticholinergic agents.

As stated earlier, biochemical and pharmacological studies indicate that the MAO enzyme exists in two forms known as “MAO Type A” (MAO-A) and “MAO Type B” (MAO-B). The forms differ in their distribution in body organs, in their substrate specificity, and in their sensitivity to inhibitors. In general, MAO-A selectively oxidizes the so-called “neurotransmitter” monoamines (epinephrine, norepinephrine and 5-hydroxytryptamine) while MAO-B selectively oxidizes the “trace” monoamines (o-tyramine, phenethylamine and tele-N-methylhistamine). Both MAO-A and MAO-B oxidize tyramine, tryptamine and dopamine. However, in man, dopamine has been shown to be a preferred substrate for MAO-B. The forms also differ in their sensitivity to inhibition, and thus, can be preferentially inhibited depending upon the chemical structure of the inhibitor and/or the relative concentrations of the inhibitor and the enzyme. The MAO inhibitors, currently available for the therapy of depression, such as tranylcypromine, phenelzine and isocarboxazid are not preferential in their action upon MAO. However, various chemical compounds are known in the art to be preferential inhibitors of MAO, the most important being clorgyline, pargyline and L-deprenyl which are all reported to be clinically effective antidepressant agents. MAO-A is preferentially inhibited by clorgyline, while MAO-B is preferentially inhibited by pargyline and 1-deprenyl. It should be observed that the “selectivity” of an MAO inhibitor arises because the inhibitor has a greater affinity for one form of the enzyme. Thus, the selectivity of an MAO inhibitor for MAO-A or MAO-B in-vivo will be dose-dependent, selectivity being lost as the dosage is increased. Clorgyline, pargyline and 1-deprenyl are selective inhibitors at lower dosages, but are not selective inhibitors at higher dosages. The literature concerning MAO-A and MAO-B, and the selective inhibition thereof, is extensive [See, for example, Goodman and Gilman, ibid, 204-205; Neff et al, Life Sciences, 14, 2061, 1974; Murphy, Biochemical Pharmacology, 27, 1889, 1978; Knoll, Chapter 10, 151-171 and Sandler, Chapter 11, 173-181, in Enzyme Inhibitors as Drugs, M. Sandler, Ed., Macmillan Press Ltd., London, 1980; Lipper et al, Psychopharmacology, 62, 123 1979; Mann et al, Life Sciences, 26, 877, 1980; various articles in Monoamines Oxidase: Structure, Function and Altered Functions, T. Singer et al, Ed., Academic Press, N.Y., 1979.

Of the selective inhibitors of MAO, 1-deprenyl is of interest since the “cheese effect” is not observed at the low dosages where preferential inhibitions of MAO-B occur. [See Knoll, TINS, 111-113, May 1979]. This observation is not unexpected since the intestinal mucosa contains predominantly MAO-A which, because it is not inhibited, permits oxidation and removal of the ingested tyramine. The selectivity of 1-deprenyl for MAO-B may account for its ability to potentiate L-DOPA for the treatment of Parkinson's disease without producing peripheral side effects, such as hypertension due to potentiation of pressor catecholamines [See Lees et al, Lancet, 791-975, Oct. 15, 1977 and Birkmeyer, Lancet, 439-443, Feb. 26, 1977].

### SUMMARY OF THE INVENTION

The present invention provides a new medical use for the compounds of formula (I), their derivatives, analogs, tautomeric forms, stereoisomers, polymorphs, hydrates, solvates, pharmaceutically acceptable salts and compositions, metabolites and prodrugs thereof, as MAO-A inhibitors.

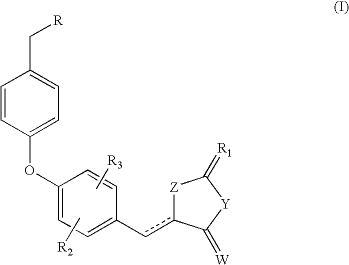

In particular the present invention preferably provides a new use for the compounds of formula (I), their derivatives, analogs, tautomeric forms, stereoisomers, polymorphs, hydrates, solvates, pharmaceutically acceptable salts and compositions, metabolites and prodrugs thereof, as MAO-A inhibitors, wherein [symbol] represents optional bond; W represents O or S; Y represents NR₄, S or O, wherein R₄ represents hydrogen, substituted or unsubstituted groups selected from alkyl, alkenyl, aryl, a counter ion or —CH₂COOR₆, wherein R₆ represents H or an alkyl group; Z represents CR₅ or S; R₁ represents O, S or together with R₅ forms a fused 5 or 6 membered aromatic or heteroaromatic ring system containing carbon atoms or 1 or 2 heteroatoms selected from O, S or N; wherein R₂, R₃, and R₅ may be same or different and independently represent hydrogen, halogens, hydroxy, nitro, cyano, formyl, amino, alkyl, haloakyl and alkoxy group.

R represents either of A, B or C; wherein A represents

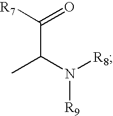

R₇ represents —OR₁₂ wherein R₁₂ represents hydrogen, substituted or unsubstituted groups selected from alkyl, alkenyl, aryl, aralkyl, heteroaryl, a counter ion or NR₁₃R₁₄, wherein R₁₃ and R₁₄ may be same or different and independently represent H, substituted or unsubstituted groups selected from alkyl, alkenyl, aryl or heteroaryl; R₈ and R₉ may be same or different and independently represent H, substituted or unsubstituted groups selected from alkyl, alkenyl, aryl, heteroaryl or COR₁₅, wherein R₁₅ represents substituted or unsubstituted groups selected from alkyl, aryl, heteroaryl, alkenyl, alkenyloxy, aryloxy, alkoxy or aralkoxy; B represents

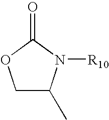

and C represents

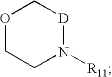

wherein, R₁₀ and R₁₁ represent H, substituted or unsubstituted groups selected from alkyl, alkenyl, aryl, aryloxy, alkoxy, heteroaryl, heterocyclyl or COR₁₆, wherein R₁₆ represents H, substituted or unsubstituted groups selected from alkyl, alkenyl, aryl, aryloxy, alkoxy or aralkoxy. Suitable groups represented by D include CH₂ or C═O.

### BRIEF DESCRIPTION OF THE FIGURES

FIG. 1 is a bar graph of MAO-A inhibition at 1 μM concentration.

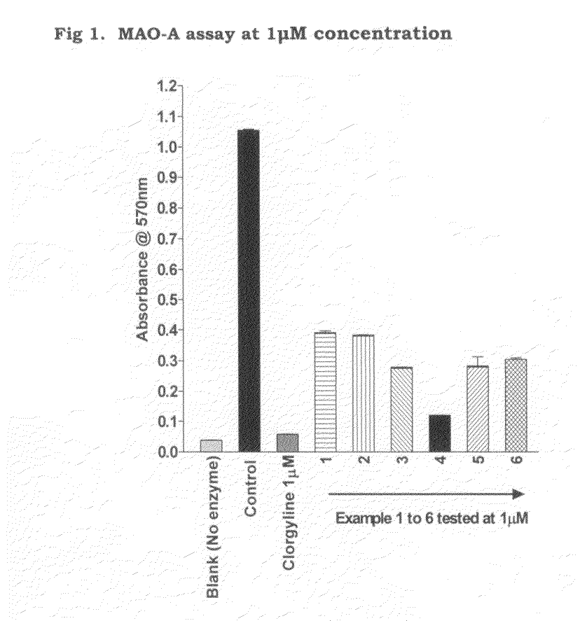

FIG. 2 is a bar graph of MAO-A inhibition at 10M concentration

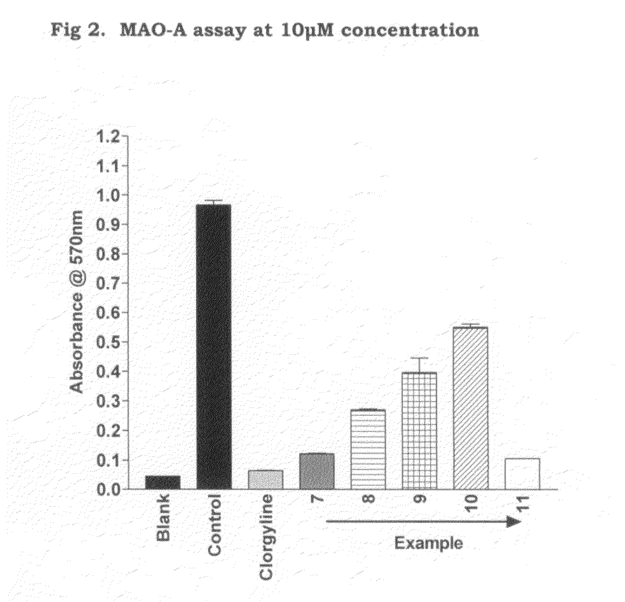

FIG. 3 is a bar graph of MAO-B inhibition at 1 μM concentration.

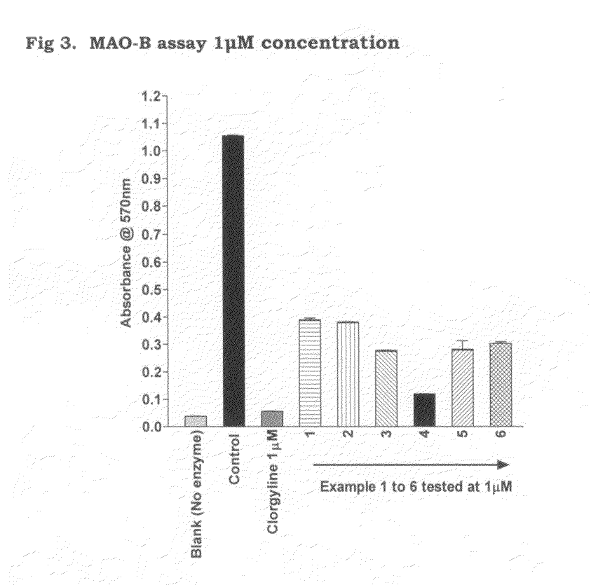

### DETAILED DESCRIPTION OF THE INVENTION

US 2005/0288341 discloses the compounds of the formula (II),

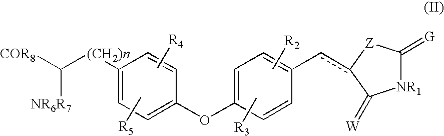

their analogs, their tautomeric forms, their stereoisomers, their polymorphs, their pharmaceutically acceptable salts, their pharmaceutically acceptable solvates, wherein [symbol] represents an optional bond; W represents O or S; Z represents CR₁₀, O or S; G represents O, S or together with R₁₀ forms a 5 or 6 membered aromatic or heteroaromatic ring system containing 1 or 2 heteroatoms selected from O, S or N.

R₂, R₃, R₄ and R₅ are selected from hydrogen, halogens such as fluorine, chlorine, bromine or iodine; hydroxy, nitro, cyano, formyl, amino, linear or branched, substituted or unsubstituted (C₁-C₆) alkyl groups such as methyl, ethyl, propyl, isopropyl, n-butyl, isobutyl, t-butyl, and the like; substituted or unsubstituted (C₁-C₆) alkoxy groups such as methoxy, ethoxy, propoxy, butoxy and the like.

R₆ and R₇ may be same or different and independently represent H, substituted or unsubstituted groups selected from alkyl, alkenyl, aryl, heteroaryl, heterocyclyl or COR₁₂, wherein R₁₂ represents substituted or unsubstituted groups selected from alkyl, alkenyl, aryl, alkenyloxy, aryloxy, alkoxy, aralkyl, or aralkoxy.

R₈ represents —OR₁₃ or NR₁₄R₁₅, wherein R₁₃ represents hydrogen, substituted or unsubstituted groups selected from alkyl, alkenyl, aryl, aralkyl, heteroaryl, or a counterion; R₁₄ and R₁₅ may be same or different and independently represent H, unsubstituted groups selected from alkyl, alkenyl and aryl.

R₁ represents hydrogen, substituted or unsubstituted groups selected from alkyl, aryl, alkenyl, a counterion, or —CH₂COOR, wherein R represents H or (C₁-C₆) alkyl.

R₁₀ optionally together with G forms a 5- or 6-membered aromatic or heteroaromatic ring system such as phenyl, furyl, pyrrolyl, pyridyl and the like. WO2006/109146 discloses the compounds of the formula (III),

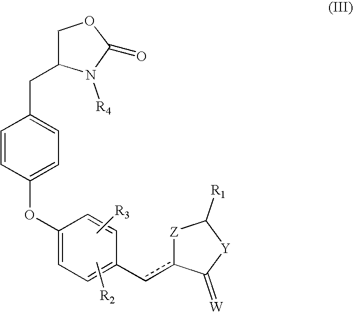

their pharmaceutically acceptable salts, wherein [symbol] represents optional bond; W represents O or S; X represents C, CH or N; Y represents NR₅, S or O, wherein R₅ represents hydrogen, substituted or unsubstituted alkyl, alkenyl, aryl, a counter ion, or —CH₂COOR, wherein R represents H or an alkyl group; Z represents CR₆ or S; R₁ represents ═O, ═S or together with R₆ forms a fused 5 or 6 membered aromatic or heteroaromatic ring system containing carbon atoms or 1 or 2 heteroatoms selected from O, S or N; R₂, R₃, may be same or different and independently represent hydrogen, halogens, hydroxy, nitro, cyano, formyl, amino, alkyl, haloakyl or alkoxy group; R₄ may be same or different and independently represent H, substituted or unsubstituted groups selected from alkyl, alkenyl, aryl, aryloxy, alkoxy, heteroaryl, heterocyclyl or COR₇, wherein R₇ represents H, substituted or unsubstituted groups selected from alkyl, alkenyl, aryl, aryloxy, alkoxy or aralkoxy. WO2006/126074 discloses the compounds of the formula (IV),

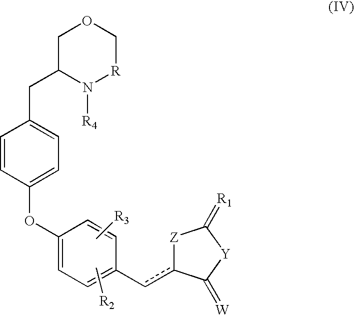

their pharmaceutically acceptable salts their analogs, their tautomeric forms, their stereoisomers and compositions; wherein [symbol] represents an optional bond; R represents CH₂, C═O; W represents O or S; X represents C, CH or N; Y represents NR₅, S or O, wherein R₅ represents hydrogen, substituted or unsubstituted alkyl, alkenyl, aryl, a counter ion or —CH₂COOR′, wherein R′ represents H or an alkyl group; Z represents CR₆ or S; R₁ represents ═O, ═S or together with R₆ forms a fused 5 or 6 membered aromatic or heteroaromatic ring system containing carbon atoms or 1 or 2 heteroatoms selected from O, S or N; R₂ and R₃ may be same or different and independently represent hydrogen, halogens, hydroxy, nitro, cyano, formyl, amino, alkyl, haloakyl, alkoxy group; R₄ represents H, substituted or unsubstituted groups selected from alkyl, alkenyl, aryl, aryloxy, alkoxy, heteroaryl, heterocyclyl, or COR₇, wherein R₇ represents H, substituted or unsubstituted groups selected from alkyl, alkenyl, aryl, aryloxy, alkoxy or aralkoxy.

These patents disclose the methods for reducing glucose, fatty acids, cholesterol and triglyceride levels in the plasma, comprising administrating an effective amount of a compound of formula (II), (III) and (IV), their analogs, their tautomers, their stereoisomers, their pharmaceutically acceptable salts and/or their pharmaceutically acceptable solvates to a patient in need thereof.

These patents disclose the methods for treating obesity, autoimmune diseases, inflammation, immunological diseases, diabetes and disorders associated with insulin resistance comprising administrating to a patient in need thereof, an effective amount of a compound of formula (II), (III) and (IV), their derivatives, analogs, tautomeric forms, stereoisomers, polymorphs, hydrates, solvates, pharmaceutically acceptable salts and compositions, metabolites and prodrugs thereof.

The present invention, provides a new medical use for the compounds of formula (II), (III) and (IV), which are disclosed in the US patent 2005/0288341, WO2006/109146 and WO2006/126074 respectively.

Compounds of formula (II), (III) and (IV), are represented by the general formula (I),

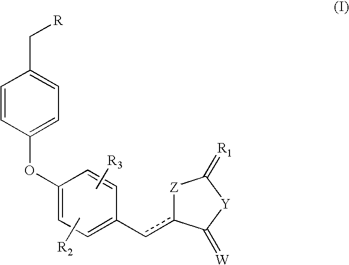

wherein, [symbol] represents optional bond;

W represents O or S;

Y represents NR₄, S or O, wherein R₄ represents hydrogen, substituted or unsubstituted groups selected from linear or branched, (C₁-C₆) alkyl groups such as methyl, ethyl, propyl, isopropyl, n-butyl, isobutyl, t-butyl and the like; alkenyl groups such as ethenyl, propenyl, butenyl and the like; aryl groups such as phenyl, naphthyl and the like, the aryl group may be substituted; a counter ion is selected from alkali metals like Li, Na, and K; or —CH₂COOR₆, wherein R₆ represents H or an alkyl group;

Z represents CR₅ or S;

R₁ is selected from O, S or together with R₅ forms a fused 5 or 6 membered aromatic or heteroaromatic ring system containing carbon atoms or 1 or 2 hetereoatoms selected form O, S or N, such as phenyl, naphthyl, furyl, pyrrolyl, pyridyl and the like.

Suitable groups represented by R₂, R₃ and R₅, are selected from hydrogen, halogens such as fluorine, chlorine, bromine or iodine; hydroxy, nitro, cyano, formyl, amino, substituted or unsubstituted linear or branched, (C₁-C₄) alkyl groups such as methyl, ethyl, propyl, isopropyl, n-butyl, isobutyl, t-butyl and the like; haloalkyl groups selected from alkyl groups substituted by one, two, three or four halogen atoms such as chloromethyl, chloroethyl, trifluoromethyl, trifluoroethyl, dichloromethyl, dichloroethyl and the like. Substituted or unsubstituted, (C₁-C₄) alkoxy groups such as methoxy, ethoxy, propoxy, butoxy and the like.

R represents either A, B or C, wherein A represents

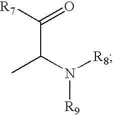

suitable groups represented by R₇ is selected from —OR₁₂, NR₁₃R₁₄.

Suitable groups represented by R₁₂ is selected from hydrogen, substituted or unsubstituted linear or branched, (C₁-C₄) alkyl groups such as methyl, ethyl, n-propyl, isopropyl, n-butyl, isobutyl, t-butyl and the like; substituted or unsubstituted linear or branched, (C₂-C₄) alkenyl groups such as ethenyl, propenyl, butenyl and the like; aryl groups such as phenyl, naphthyl and the like, the aryl group may be substituted; aralkyl groups such as benzyl, phenyl ethyl, phenyl propyl and the like, which may be substituted; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl, quinolinyl and the like, which may be substituted; the counter ion is selected from alkali metals like Li, Na, and K; alkaline earth metals like Ca and Mg; salts of different bases such as ammonium or substituted ammonium salts, diethanolamine, α-phenylethylamine, benzylamine, piperidine, morpholine, pyridine, hydroxyethylpyrrolidine, hydroxyethylpiperidine, choline and the like, aluminum, tromethamine and the like.

Suitable groups represented by R₁₃ and R₁₄ are selected from hydrogen, substituted or unsubstituted linear or branched, (C₁-C₄) alkyl groups such as methyl, ethyl, n-propyl, isopropyl, n-butyl, isobutyl, t-butyl and the like; substituted or unsubstituted linear or branched, (C₂-C₂₀) alkenyl groups such as ethenyl, propenyl, butenyl and the like; aryl groups such as phenyl, naphthyl and the like, the aryl group may be substituted; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl, quinolinyl and the like.

Suitable groups represented by R₈ and R₉ are selected from H, COR₁₅, substituted or unsubstituted linear or branched, (C₁-C₄) alkyl groups such as methyl, ethyl, n-propyl, isopropyl, n-butyl, isobutyl, t-butyl and the like; Substituted or unsubstituted linear or branched, (C₂-C₄) alkenyl groups such as ethenyl, propenyl, butenyl and the like; aryl groups such as phenyl, naphthyl and the like, the aryl group may be substituted; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl and the like, which may be substituted.

Suitable groups represented by R₁₅ are selected from hydrogen, substituted or unsubstituted linear or branched, (C₁-C₄) alkyl groups such as methyl, ethyl, n-propyl, isopropyl, n-butyl, isobutyl, t-butyl and the like; substituted or unsubstituted linear or branched, (C₂-C₄) alkenyl groups such as ethenyl, propenyl, butenyl and the like; aryl groups such as phenyl, naphthyl and the like, the aryl group may be substituted; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl, quinolinyl and the like; alkoxy groups such as methoxy, ethoxy, n-propoxy, isopropoxy and the like, which may be substituted; aralkoxy groups such as phenylmethoxy, phenylethoxy, phenylpropoxy and the like; aryloxy groups such as phenoxy, napthoxy and the like.

B represents

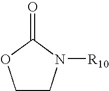

and C represents

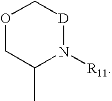

Suitable groups represented by R₁₀ and R₁₁ are selected from H, alkyl, alkenyl, substituted or unsubstituted, (C₁-C₄) alkyl groups such as methyl, ethyl, propyl, isopropyl, n-butyl, isobutyl, t-butyl and the like; substituted or unsubstituted linear or branched, (C₂-C₇) alkenyl groups such as ethenyl, propenyl, butenyl and the like; aryl groups such as phenyl, naphthyl and the like, the aryl group may be substituted; aryloxy groups such as phenoxy, napthoxy and the like, substituted or unsubstituted linear or branched, (C₂-C₄) alkoxy groups such as methoxy, ethoxy, propoxy, n-butoxy and the like; heteroaryl groups such as pyridyl, thienyl, furyl, pyrrolyl, oxazolyl, thiazolyl, imidazolyl, oxadiazolyl, triazolyl, tetrazolyl, pyrimidinyl, pyrazinyl, indolyl, indolinyl, benzothiazolyl and the like, which may be substituted; heterocyclyl groups such as pyrrolidinyl, thiazolidinyl, oxazolidinyl, morpholinyl, thiomorpholinyl, piperidinyl, piperazinyl and the like, which may be substituted, COR₁₆, wherein R₁₆ represents H; substituted or unsubstituted, (C₁-C₄) alkyl groups such as methyl, ethyl, propyl, isopropyl, n-butyl, isobutyl, t-butyl and the like; substituted or unsubstituted linear or branched, (C₂-C₇) alkenyl groups such as ethenyl, propenyl, butenyl and the like; aryl groups such as phenyl, naphthyl and the like, the aryl group may be substituted; aryloxy groups such as phenoxy, napthoxy and the like; substituted or unsubstituted linear or branched, (C₂-C₂₀) alkoxy groups such as methoxy, ethoxy, propoxy, n-butoxy, isobutoxy, t-butoxy and the like. Suitable groups represented by D include CH₂ or C═O.

Suitable substitutents are selected from halogens such as fluorine, chlorine, bromine or iodine, hydroxy, nitro, cyano, azido, nitroso, amino, hydrazine, hydrazide, hydroxamate, formyl, alkyl, haloalkyl, haloalkoxy, cycloalkyl, aryl, alkoxy, aryloxy, acyl, acyloxy, acyloxyacyl, heterocyclyl, heteroaryl, monoalkylamino, dialkylamino, acylamino, alkylaminocarbonyl, alkoxycarbonyl, aryloxycarbonyl, heterocyclylcarbonyl, arylsulfonyl, alkylsulfinyl, arylsulfinyl, alkylthio, arylthio, sulfamoyl, alkoxyalkyl groups and carboxylic acids or its derivatives such as carboxamide and carboxamidoalkyl.

The term analog includes a compound, which differs from the parent structure by one or more C, N, O or S atoms. Hence, a compound in which one of the N atoms in the parent structure is replaced by an S atom is an analog of the former.

The term stereoisomer includes isomers that differ from one another in the way the atoms are arranged in space, but whose chemical formulas and structures are otherwise identical. Stereoisomers include enantiomers and diastereoisomers.

The term tautomers include readily interconvertible isomeric forms of a compound in equilibrium. The enol-keto tautomerism is an example.

The term polymorphs include crystallographically distinct forms of compounds with chemically identical structures.

The term pharmaceutically acceptable solvates includes combinations of solvent molecules with molecules or ions of the solute compound.

The term derivative refers to a compound obtained from a compound according to formula (I), an analog, tautomeric form, stereoisomer, polymorph, hydrate, pharmaceutically acceptable salt or pharmaceutically acceptable solvate thereof, by a simple chemical process converting one or more functional groups, such as, by oxidation, hydrogenation, alkylation, esterification, halogenation and the like.

Pharmaceutically acceptable salts of the present invention include alkali metal like Li, Na, and K, alkaline earth metal like Ca and Mg, salts of organic bases such as diethanolamine, α-phenylethylamine, benzylamine, piperidine, morpholine, pyridine, hydroxyethylpyrrolidine, hydroxyethylpiperidine, choline and the like, ammonium or substituted ammonium salts, aluminum salts. Salts also include amino acid salts such as glycine, alanine, cystine, cysteine, lysine, arginine, phenylalanine, guanidine etc. Salts may include acid addition salts where appropriate which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, trifluoroacetates, ascorbates, glycerophosphates, ketoglutarates and the like. Pharmaceutically acceptable solvates may be hydrates or comprising other solvents of crystallization such as alcohols.

A term once described, the same meaning applies for it, throughout the patent

Recently we have discovered that the compounds of formula (I) are able to inhibit the MAO-A enzyme and are useful in the prophylaxis and treatment of mental disorders such as depression.

Depression states in which the compounds are particularly useful are those defined in the Diagnostic and Statistical Manual of Mental Disorders, third edition (DSM III), American Psychiatric Association, Washington, D.C., 1980, (DSM III, 296.2X to 296.6X and 301.13), including that characterized by anxiety or obsessional neuroses (DSM III, 300.40), or atypical depression including depression in the elderly or symptoms of early senility, especially symptoms relating to sociability and quality of life (DSM III, 296.70 and 296.82), e.g., accompanied by a personality disorder.

### Particularly Preferred Compounds of the Present Invention Include:

1. Methyl 2-amino-3-(4-{4-[(4-oxo-2-thioxo-1,3-thiazolidin-5-yl)methyl]phenoxy}phenyl)propanoate hydrochloride;
2. Methyl (2S)-2-amino-3-(4-{4-[(4-oxo-2-thioxo-1,3-thiazolidin-5-yl)methyl]phenoxy}phenyl)propanoate hydrochloride;
3. Methyl (2S)-2-amino-3-(4-{4-[(2,4-dioxo-1,3-thiazolidin-5-yl)methyl]-2-fluorophenoxy}phenyl)propanoate hydrochloride;
4. 3-(2-Chloro-4-{4-[(3-methyl-2-oxo-1,3-oxazolidin-4-yl)methyl]phenoxy benzylidene)-1,3-dihydro-2H-indol-2-one;
5. 3-(2-Chloro-4-{4-[(3-ethyl-2-oxo-1,3-oxazolidin-4-yl)methyl]phenoxy}benzylidene)-1,3-dihydro-2H-indol-2-one;
6. 3-(2-Chloro-4-{4-[(5-oxomorpholin-3-yl)methyl]phenoxy}benzylidene)-1,3-dihydro-2H-indol-2-one;
7. Methyl (2S)-2-amino-3-(4-{3-chloro-4-[(2-oxo-1,2-dihydro-3H-indol-3-ylidene)methyl]phenoxy}phenyl)propanoate hydrochloride;
8. Methyl (2S)-2-amino-3-(4-{2-chloro-4-[(2,4-dioxo-1,3-thiazolidin-5-yl)methyl]-phenoxy}phenyl)propanoate hydrochloride;
9. [4-oxo-5-(4-{4-[(3-ethyl-2-oxo-1,3-oxazolidin-4-yl)methyl]phenoxy}benzyl)-2-thioxo-1,3-thiazolidin-3-yl]acetic acid;
10. 4-(4-{2-chloro-4-[(4-oxo-2-thioxo-1,3-thiazolidin-5-yl)methyl]phenoxy}benzyl)-3-methyl-1,3-oxazolidin-2-one and
11. Methyl(2S)-2-amino-3-(4-{4-[(2,4-dioxo-1,3-thiazolidin-5-yl)methyl]phenoxy}phenyl)propanoate hydrochloride.

The pharmaceutically acceptable salts are prepared by reacting the compound of formula (I) with 1 to 10 equivalents of a base such as sodium hydroxide, sodium methoxide, sodium hydride, potassium t-butoxide, calcium hydroxide, magnesium hydroxide and the like, in solvents like ether, tetrahydrofuran, methanol, t-butanol, dioxane, isopropanol, ethanol etc. Mixture of solvents may also be used. Organic bases such as diethanolamine, α-phenylethylamine, benzylamine, piperidine, morpholine, pyridine, hydroxyethylpyrrolidine, hydroxyethylpiperidine, choline, guanidine and the like, ammonium or substituted ammonium salts, aluminum salts. Amino acids such as glycine, alanine, cystine, cysteine, lysine, arginine, phenylalanine etc may be used for the preparation of amino acid salts. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid, salicylic acid, hydroxynaphthoic acid, ascorbic acid, palmitic acid, succinic acid, benzoic acid, benzenesulfonic acid, tartaric acid, oxalic acid and the like in solvents like ethyl acetate, ether, alcohols, acetone, tetrahydrofuran, dioxane etc. Mixture of solvents may also be used.

It should be noted that compounds of the invention may contain groups that may exist in tautomeric forms, and though one form is named, described, displayed and/or claimed herein, all the forms are intended to be inherently included in such name, description, display and/or claim.

The stereoisomers of the compounds forming part of this invention may be prepared by using reactants in their single enantiomeric form, in the process wherever possible or by conducting the reaction in the presence of reagents or catalysts in their single enantiomeric form or by resolving the mixture of stereoisomers by conventional methods. Some of the preferred methods include use of microbial resolution, resolving the diastereomeric salts formed with chiral acids such as mandelic acid, camphorsulfonic acid, tartaric acid, lactic acid, and the like, wherever applicable or by using chiral bases such as brucine, cinchona alkaloids, their derivatives and the like.

Prodrugs of the compounds of formula (I) are also contemplated by this invention. A prodrug is an active or inactive compound that is modified chemically through in-vivo physiological action, such as hydrolysis, metabolism and the like, into a compound of this invention following administration of the prodrug to a patient. The suitability and techniques involved in making and using prodrugs are well known by those skilled in the art.

Various polymorphs of the compounds of the general formula (I), forming part of this invention may be prepared by crystallization of the compounds of formula (I) under different conditions. For example, using different commonly used solvents, or their mixtures for recrystallization; crystallizations at different temperatures; various modes of cooling, ranging from very fast to very slow cooling during crystallizations. Heating or melting the compounds followed by cooling gradually or immediately, one can also obtain polymorphs. The presence of polymorphs may be determined by solid probe NMR spectroscopy, IR spectroscopy, differential scanning calorimetry and powder X-ray diffraction or other such techniques.

Pharmaceutically acceptable solvates of the compounds of the formula (I) forming part of this invention may be prepared by conventional methods such as dissolving the compounds of the formula (I) in solvents such as water, methanol, ethanol, mixture of solvents such as acetone:water, dioxane:water, N,N-dimethylformamide:water and the like, preferably water and recrystallization by using different crystallization techniques.

The present invention also provides a pharmaceutical composition, containing one or more of the compounds of the general formula (I) as defined above, their derivatives, analogs, tautomeric forms, stereoisomers, polymorphs, hydrates, metabolites, prodrugs, pharmaceutically acceptable salts, pharmaceutically acceptable solvates in combination with the usual pharmaceutically employed carriers, diluents and the like, useful for the treatment of and/or prophylaxis of mental disorders such as depression in a mammal.

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. The compositions may be prepared by processes known in the art. The amount of the active ingredient in the composition may be less than 70% by weight. Such compositions typically contain from 1 to 25%, preferably 1 to 15% by weight of active compound, the remainder of the composition being pharmaceutically acceptable carriers, diluents, excipients or solvents.

Suitable pharmaceutically acceptable carriers include solid fillers or diluents and sterile aqueous or organic solutions. The active compound will be present in such pharmaceutical compositions in the amounts sufficient to provide the desired dosage in the range as described above. Thus, for oral administration, the compounds can be combined with a suitable solid or liquid carrier or diluent to form capsules, tablets, powders, syrups, solutions, suspensions and the like. The pharmaceutical compositions, may, if desired, contain additional components such as flavorants, sweeteners, excipients and the like. For parenteral administration, the compounds can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or alkali or alkaline earth metal salts of the compounds. The injectable solutions prepared in this manner can then be, administered intravenously, intraperitoneally, subcutaneously, or intramuscularly, with intramuscular administration being preferred in humans.

The pharmaceutical compositions of the invention are effective in treating a disease, condition or disorder modulated by a cannabinoid receptor antagonist in animals, wherein said disease, condition or disorder modulated by a cannabinoid receptor antagonist is selected from the group consisting of weight loss, obesity, bulimia, depression, bipolar disorders, psychoses, schizophrenia, behaviors, alcoholism, tobacco abuse, memory loss, Alzheimer's disease, dementia of aging, seizure disorders, epilepsy, attention deficit disorder and Parkinson's disease.

Generally, the effective dose for treating a particular condition in a patient may be readily determined and adjusted by the physician during treatment to alleviate the symptoms or indications of the condition or disease. Generally, a daily dose of active compound in the range of about 0.01 to 1000 mg/kg of body weight is appropriate for administration to obtain effective results. The daily dose may be administered in a single dose or divided into several doses. In some cases, depending upon the individual response, it may be necessary to deviate upwards or downwards from the initially prescribed daily dose. Typical pharmaceutical preparations normally contain from about 0.2 to about 500 mg of active compound of formula I and/or its pharmaceutically active salts or solvates per dose.

While the compounds of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition.

The term “therapeutically effective amount” or “effective amount” refers to that amount of a compound or mixture of compounds of Formula I that is sufficient to effect treatment, as defined below, when administered alone or in combination with other therapies to a mammal in need of such treatment.

The term “animal” as used herein is meant to include all mammals, and in particular humans. Such animals are also referred to herein as subjects or patients in need of treatment. The therapeutically effective amount will vary depending upon the subject and disease condition being treated, the weight and age of the subject, the severity of the disease condition, the particular compound of Formula I chosen, the dosing regimen to be followed, timing of administration, the manner of administration and the like, all of which can readily be determined by one of ordinary skill in the art.

The term “treatment” or “treating” means any treatment of a disease in a mammal, including:
a) Preventing the disease, that is, causing the clinical symptoms of the disease not to develop;
b) Inhibiting the disease, that is, slowing or arresting the development of clinical symptoms; and/or
c) Relieving the disease, that is, causing the regression of clinical symptoms.

From the foregoing description, one skilled in the art can easily ascertain the essential characteristics of this invention, and without departing from the spirit and scope thereof, make various changes and modifications of the invention to adapt it to various usages and conditions.

The present invention is provided by the examples given below, which are provided by the way of illustration only, and should not be construed to limit the scope of the invention. Variation and changes, which are obvious to one skilled in the art, are intended to be within the scope and nature of the invention, which are defined in the appended claims.

Structures of the Compounds within the Scope of the Invention are Provided Below in the Table I:

TABLE I

| Compound | |
|---|---|
| No. | Structure |
| 1 | 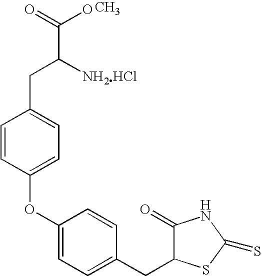 |
| 2 | 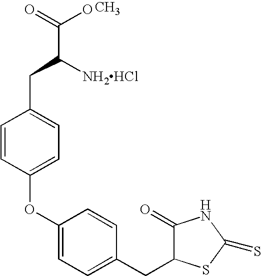 |
| 3 | 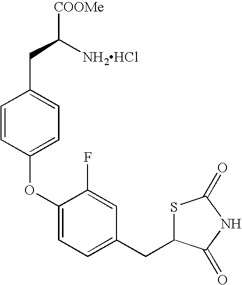 |
| 4 | 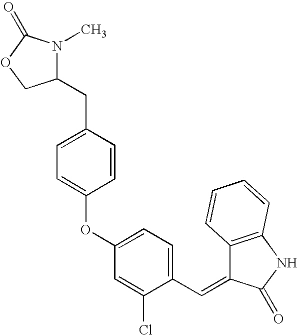 |
| 5 | 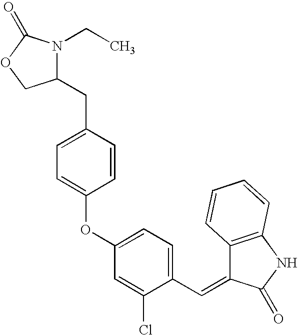 |
| 6 | 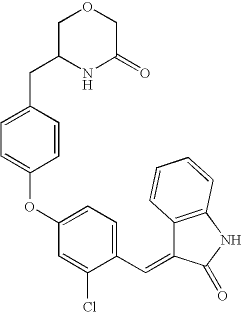 |
| 7 | 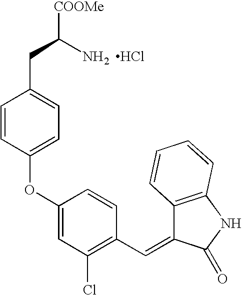 |
| 8 | 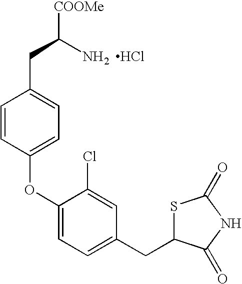 |
| 9 | 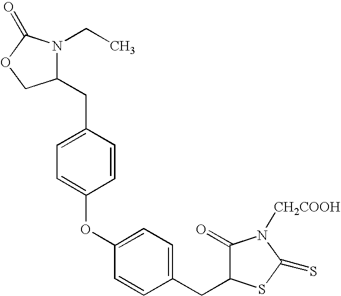 |
| 10 | 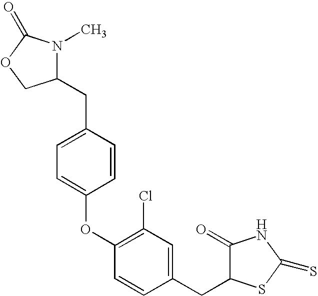 |
| 11 | 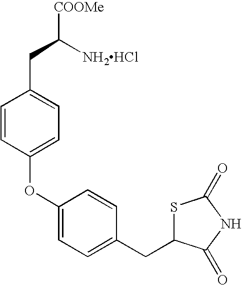 |

### Protocols for Biology Testing

Monoamine oxidase (MAO) is a flavin-containing enzyme that catalyses the oxidation of a variety of amine-containing neurotransmitters such as serotonin, nor epinephrine, epinephrine and dopamine to yield the corresponding aldehydes. MAO assay kit (Cat no# FLMAO 100-3) was purchased from Cell Technology Inc, Mountain View, Calif. The pure MAO-A (Cat no# 456283) was purchased from BD biosciences, Woburn, Mass. The MAO-B enzyme (Cat no# M7441) was purchased from Sigma Aldrich, St Louis, Mo. The MAO A and B assay procedures were followed in as described in the protocol. In the MAO A and B assay, 1× reaction buffer (part# 3020, Cell tech, Mountain View, Calif.), HRP (Part # 6007, Cell tech), Detection Reagent (Part # 4009, Cell tech), Substrate: Bezylamine (Part # 7006, Cell tech), Tyramine (Part # 7005, Cell tech) were used. MAO-A inhibitor (Clorgyline, Part # 7002, Cell tech) and MAO-B inhibitor (Deprenyl, Cat # 086K4710, Sigma Aldrich, St Louis, Mo.) were used to test the monoamine oxidase activity. The reaction cocktail was prepared by adding 100 μl of detection reagent, 100 μl of 100×HRP, 100 μl of substrate and 9.7 ml of 1× reaction buffer. Tyramine was used as a substrate and Clorgyline as an inhibitor for the MAO-A assay. For MAO-B assay, benzyl amine and deprenyl were used as the substrate and inhibitor respectively. The kynuramine deamination activity of MAO-A enzyme was reported (BD Biosciences, MA) as 81 Units/mg protein. The MAO-A enzyme titration was carried out and it was found out that 1 unit/ml protein concentration was optimum to carry out the assay. Similarly the kynuramine deamination activity of MAO-B enzyme was reported as 38-units/mg protein. The MAO-B enzyme titration was carried out and it was found out that 1 unit/ml protein concentration was optimum to carry out the assay. To begin with the assay initially 1× reaction buffer was added to all the wells. The MAO-A or B enzyme and their respective inhibitors and all unknown compounds were added individually to each well. The plate was then incubated at room temperature for 30 minutes for the enzyme and inhibitor interaction. After 30 minutes of pre-incubation, 100 μl of reaction cocktail mixture was added to all the wells and the reading was taken in the molecular device (Emax) ELISA plate reader at 570 nm from 0-60 minutes with a 5 minutes interval.

1. A compound of formula (I), 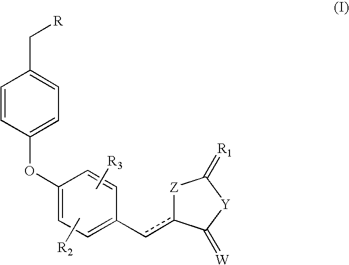
its derivatives, analogs, tautomeric forms, stereoisomers, polymorphs, hydrates, solvates, pharmaceutically acceptable salts and compositions, metabolites and prodrugs thereof, wherein, [symbol] represents optional bond;
W represents O or S;
Y represents NR₄, S or O, wherein R₄ represents hydrogen, substituted or unsubstituted groups selected from linear or branched (C₁-C₆) alkyl groups comprising methyl, ethyl, propyl, isopropyl, n-butyl, isobutyl and t-butyl; alkenyl groups comprising ethenyl, propenyl and butenyl; aryl groups comprising phenyl and naphthyl, the aryl group is optionally substituted; a counter ion is selected from alkali metals like Li, Na, and K; or-CH₂COOR_{6,} wherein R₆ represents H or an alkyl group;
Z represents CR₅ or S;
R₁ is selected from O, S or together with R₅ forms a fused 5 or 6 membered aromatic or heteroaromatic ring system containing carbon atoms or 1 or 2 hetereoatoms selected form O, S or N, comprising phenyl, naphthyl, furyl, pyrrolyl and pyridyl;
suitable groups represented by R₂, R₃ and R₅, are selected from hydrogen, halogens comprising fluorine, chlorine, bromine or iodine; hydroxy, nitro, cyano, formyl, amino, substituted or unsubstituted linear or branched (C₁-C₄) alkyl groups comprising methyl, ethyl, propyl, isopropyl, n-butyl, isobutyl and t-butyl; haloalkyl groups selected from alkyl groups substituted by one, two, three or four halogen atoms comprising chloromethyl, chloroethyl, trifluoromethyl, trifluoroethyl, dichloromethyl and dichloroethyl; substituted or unsubstituted, (C₁-C₄) alkoxy groups comprising methoxy, ethoxy, propoxy and butoxy;
R represents either A, B or C, wherein A represents 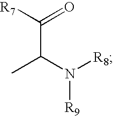
suitable groups represented by R₇ is selected from —OR₁₂, NR₁₃R₁₄; suitable groups represented by R₁₂ is selected from hydrogen, substituted or unsubstituted, linear or branched, (C₁-C₄) alkyl groups comprising methyl, ethyl, n-propyl, isopropyl, n-butyl, isobutyl and t-butyl; substituted or unsubstituted linear or branched, (C₂-C₄) alkenyl groups comprising ethenyl, propenyl and butenyl; aryl groups comprising phenyl and naphthyl, the aryl group is optionally substituted; aralkyl groups comprising benzyl, phenylethyl and phenylpropyl which are optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl and quinolinyl which are optionally substituted; the counter ion is selected from alkali metals like Li, Na, and K; alkaline earth metals like Ca and Mg; salts of different bases comprising ammonium or substituted ammonium salts, diethanolamine, α-phenylethylamine, benzylamine, piperidine, morpholine, pyridine, hydroxyethylpyrrolidine, hydroxyethylpiperidine, choline, aluminum and tromethamine;
suitable groups represented by R₁₃ and R₁₄ are selected from hydrogen, substituted or unsubstituted linear or branched, (C₁-C₄) alkyl groups comprising methyl, ethyl, n-propyl, isopropyl, n-butyl, isobutyl and t-butyl; substituted or unsubstituted linear or branched, (C₂-C₂₀) alkenyl groups comprising ethenyl, propenyl and butenyl; aryl groups comprising phenyl and naphthyl, the aryl group is optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl and quinolinyl;
suitable groups represented by R₈ and R₉ are selected from H, COR₁₅, substituted or unsubstituted linear or branched, (C₁-C₄) alkyl groups comprising methyl, ethyl, n-propyl, isopropyl, n-butyl, isobutyl and t-butyl; substituted or unsubstituted linear or branched, (C₂-C₄) alkenyl groups comprising ethenyl, propenyl and butenyl; aryl groups comprising phenyl and naphthyl, the aryl group is optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl and pyridazinyl which are optionally substituted;
suitable groups represented by R₁₅ are selected from hydrogen, substituted or unsubstituted linear or branched, (C₁-C₄) alkyl groups comprising methyl, ethyl, n-propyl, isopropyl, n-butyl, isobutyl and t-butyl; substituted or unsubstituted linear or branched, (C₂-C₄) alkenyl groups comprising ethenyl, propenyl and butenyl; aryl groups comprising phenyl and naphthyl, the aryl group is optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl and quinolinyl; alkoxy groups comprising methoxy, ethoxy, n-propoxy and isopropoxy, which are optionally substituted; aralkoxy groups comprising phenylmethoxy, phenylethoxy and phenylpropoxy; aryloxy groups comprising phenoxy and napthoxy;
B represents 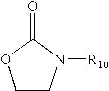
and C represents 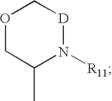
suitable groups represented by R₁₀ and R₁₁ are selected from H, alkyl, alkenyl, substituted or unsubstituted, (C₁-C₄) alkyl groups comprising methyl, ethyl, propyl, isopropyl, n-butyl, isobutyl and t-butyl; substituted or unsubstituted linear or branched, (C₂-C₇) alkenyl groups comprising ethenyl, propenyl and butenyl; aryl groups comprising phenyl and naphthyl, the aryl group is optionally substituted; aryloxy groups comprising phenoxy and napthoxy substituted or unsubstituted linear or branched, (C₂-C₄) alkoxy groups comprising methoxy, ethoxy, propoxy and n-butoxy; heteroaryl groups comprising pyridyl, thienyl, furyl, pyrrolyl, oxazolyl, thiazolyl, imidazolyl, oxadiazolyl, triazolyl, tetrazolyl, pyrimidinyl, pyrazinyl, indolyl, indolinyl and benzothiazolyl which are optionally substituted; heterocyclyl groups comprising pyrrolidinyl, thiazolidinyl, oxazolidinyl, morpholinyl, thiomorpholinyl, piperidinyl and piperazinyl which are optionally substituted, COR₁₆, wherein R₁₆ represents H; substituted or unsubstituted, (C₁-C₄) alkyl groups comprising methyl, ethyl, propyl, isopropyl, n-butyl, isobutyl and t-butyl; substituted or unsubstituted linear or branched, (C₂-C₇) alkenyl groups comprising ethenyl, propenyl and butenyl; aryl groups comprising phenyl and naphthyl, the aryl group is optionally substituted; aryloxy groups comprising phenoxy and napthoxy; substituted or unsubstituted linear or branched, (C₂-C₂₀) alkoxy groups comprising methoxy, ethoxy, propoxy, n-butoxy, isobutoxy and t-butoxy; suitable groups represented by D include CH₂ or C=O;
suitable substitutents are selected from halogens comprising fluorine, chlorine, bromine or iodine; hydroxy, nitro, cyano, azido, nitroso, amino, hydrazine, hydrazide, hydroxamate, formyl, alkyl, haloalkyl, haloalkoxy, cycloalkyl, aryl, alkoxy, aryloxy, acyl, acyloxy, acyloxyacyl, heterocyclyl, heteroaryl, monoalkylamino, dialkylamino, acylamino, alkylaminocarbonyl, alkoxycarbonyl, aryloxycarbonyl, heterocyclylcarbonyl, arylsulfonyl, alkylsulfinyl, arylsulfinyl, alkylthio, arylthio, sulfamoyl, alkoxyalkyl groups and carboxylic acids or its derivatives comprising carboxamide and carboxamidoalkyl.

2. A compound of formula (I) as claimed in claim 1, which is selected from the group consisting of:
Methyl 2-amino-3-(4-{4-[(4-oxo-2-thioxo-1,3-thiazolidin-5- yl)methyl]phenoxy}phenyl)propanoate hydrochloride;
Methyl (2S)-2-amino-3-(4-{4-[(4-oxo-2-thioxo- 1,3-thiazolidin-5-yl)methyl]phenoxy}phenyl)propanoate hydrochloride;
Methyl (2S)-2-amino-3-(4-{4- [(2,4-dioxo-1,3-thiazolidin-5-yl)methyl]-2- fluorophenoxy}phenyl)propanoate hydrochloride;
3-(2-Chloro-4-{4-[(3-methyl-2-oxo-1,3-oxazolidin-4-yl)methyl]phenoxy benzylidene)- 1,3-dihydro-2H-indol-2-one;
3-(2-Chloro-4-{4-[(3-ethyl-2-oxo-1,3-oxazolidin-4-yl) methyl]phenoxy}benzylidene)- 1,3-dihydro-2H-indol-2-one;
3-(2-Chloro-4-{4- [(5-oxomorpholin-3-yl)methyl]phenoxy}benzylidene) -1,3-dihydro-2H-indol-2-one;
Methyl (2S)-2-amino-3-(4-{3-chloro-4-[(2-oxo- 1,2-dihydro-3H-indol-3-ylidene) methyl]phenoxy}phenyl)propanoate hydrochloride;
Methyl (2S)-2-amino-3-(4-{2-chloro-4-[(2,4-dioxo- 1,3-thiazolidin-5- yl)methyl]- phenoxy}phenyl)propanoate hydrochloride;
[4-oxo-5-(4-{4-[(3-ethyl-2-oxo-1,3-oxazolidin-4-yl)methyl]phenoxy}benzyl)- 2-thioxo- 1,3-thiazolidin-3-yl]acetic acid;
4-(4-{2-chloro-4-[(4-oxo-2-thioxo- 1,3-thiazolidin-5-yl)methyl]phenoxy}benzyl)-3-methyl- 1,3-oxazolidin-2-one and
Methyl(25)-2-amino-3 -(4-{4-[(2,4-dioxo-1,3-thiazolidin-5- yl)methyl]phenoxy}phenyl)propanoate hydrochloride.

3. A pharmaceutical composition comprising a compound of formula (I) as claimed in claim 1, or a pharmaceutically acceptable salt thereof, or a solvate or hydrate of said compound or said salt, as an active ingredient along with a pharmaceutically acceptable carrier, diluent, excipient or solvate.

4. A pharmaceutical composition as claimed in claim 3, wherein the amount of the compound of formula (I) in the composition is less than 70% by weight.

5. A pharmaceutical composition as claimed in claim 3, in the form of a tablet, capsule, powder, syrup, solution, aerosol or suspension.

6-8. (canceled)

9. A method for inhibiting the MAO enzyme for the treatment of a mental disorder comprising administering, to a subject in need thereof of a therapeutically effective amount of a compound of formula (I), 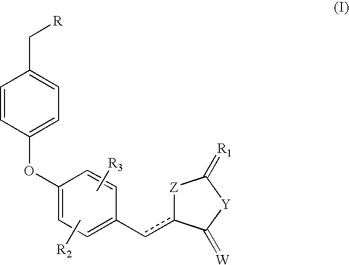
its derivatives, analogs, tautomeric forms, stereoisomers, polymorphs, hydrates, solvates, pharmaceutically acceptable salts, metabolites and prodrugs thereof, wherein [symbol] represents optional bond;
W represents O or S;
Y represents NR_{4, S or O, wherein R}₄ represents hydrogen, substituted or unsubstituted groups selected from linear or branched (C₁-C₆) alkyl groups comprising methyl, ethyl, propyl, isopropyl, n-butyl, isobutyl and t-butyl; alkenyl groups comprising ethenyl, propenyl and butenyl; aryl groups comprising phenyl and naphthyl, the aryl group is optionally substituted; a counter ion is selected from alkali metals like Li, Na, and K; or - CH₂COOR₆, wherein R₆ represents H or an alkyl group;
Z represents CR₅ or S;
R₁ is selected from O, S or together with R₅ forms a fused 5 or 6 membered aromatic or heteroaromatic ring system containing carbon atoms or 1 or 2 hetereoatoms selected form O, S or N, comprising phenyl, naphthyl, furyl, pyrrolyl and pyridyl;
suitable groups represented by R₂, R₃ and R₅, are selected from hydrogen, halogens comprising fluorine, chlorine, bromine or iodine; hydroxy, nitro, cyano, formyl, amino, substituted or unsubstituted linear or branched (C₁-C₄) alkyl groups comprising methyl, ethyl, propyl, isopropyl, n-butyl, isobutyl and t-butyl; haloalkyl groups selected from alkyl groups substituted by one, two, three or four halogen atoms comprising chloromethyl, chloroethyl, trifluoromethyl, trifluoroethyl, dichloromethyl and dichloroethyl; substituted or unsubstituted, (C₁-C₄) alkoxy groups comprising methoxy, ethoxy, propoxy and butoxy;
R represents either A, B or C, wherein A represents 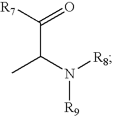
suitable groups represented by R₇ is selected from —OR₁₂, NR₁₃R₁₄; suitable groups represented by R₁₂ is selected from hydrogen, substituted or unsubstituted, linear or branched, (C₁-C₄) alkyl groups comprising methyl, ethyl, n-propyl, isopropyl, n-butyl, isobutyl and t-butyl; substituted or unsubstituted linear or branched, (C₂-C₄) alkenyl groups comprising ethenyl, propenyl and butenyl; aryl groups comprising phenyl and naphthyl, the aryl group is optionally substituted; aralkyl groups comprising benzyl, phenylethyl and phenylpropyl which are optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl and quinolinyl which are optionally substituted; the counter ion is selected from alkali metals like Li, Na, and K; alkaline earth metals like Ca and Mg; salts of different bases comprising ammonium or substituted ammonium salts, diethanolamine, α-phenylethylamine, benzylamine, piperidine, morpholine, pyridine, hydroxyethylpyrrolidine, hydroxyethylpiperidine, choline, aluminum and tromethamine;
suitable groups represented by R₁₃ and R₁₄ are selected from hydrogen, substituted or unsubstituted linear or branched, (C₁-C₄) alkyl groups comprising methyl, ethyl, n-propyl, isopropyl, n-butyl, isobutyl and t-butyl; substituted or unsubstituted linear or branched, (C₂-C₂₀) alkenyl groups comprising ethenyl, propenyl and butenyl; aryl groups comprising phenyl and naphthyl, the aryl group is optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl and quinolinyl;
suitable groups represented by R₈ and R₉ are selected from H, COR₁₅, substituted or unsubstituted linear or branched, (C₁-C₄) alkyl groups comprising methyl, ethyl, n-.propyl, isopropyl, n-butyl, isobutyl and t-butyl; substituted or unsubstituted linear or branched, (C₂-C₄) alkenyl groups comprising ethenyl, propenyl and butenyl; aryl groups comprising phenyl and naphthyl, the aryl group is optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl and pyridazinyl which are optionally substituted;
suitable groups represented by R₁₅ are selected from hydrogen, substituted or unsubstituted linear or branched, (C₁-C₄) alkyl groups comprising methyl, ethyl, n-propyl, isopropyl, n-butyl, isobutyl and t-butyl; substituted or unsubstituted linear or branched, (C₂-C₄) alkenyl groups comprising ethenyl, propenyl and butenyl; aryl groups comprising phenyl and naphthyl, the aryl group is optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl and quinolinyl; alkoxy groups comprising methoxy, ethoxy, n-propoxy and isopropoxy, which are optionally substituted; aralkoxy groups comprising phenylmethoxy, phenylethoxy and phenylpropoxy; aryloxy groups comprising phenoxy and napthoxy; B represents 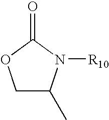
and C represents 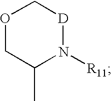
suitable groups represented by R₁₀ and R₁₁ are selected from H, alkyl, alkenyl, substituted or unsubstituted, (C₁-C₄) alkyl groups comprising methyl, ethyl, propyl, isopropyl, n-butyl, isobutyl and t-butyl; substituted or unsubstituted linear or branched, (C₂-C₇) alkenyl groups comprising ethenyl, propenyl and butenyl; aryl groups comprising phenyl and naphthyl, the aryl group is optionally substituted; aryloxy groups comprising phenoxy and napthoxy substituted or unsubstituted linear or branched, (C₂-C₄) alkoxy groups comprising methoxy, ethoxy, propoxy and n-butoxy; heteroaryl groups comprising pyridyl, thienyl, furyl, pyrrolyl, oxazolyl, thiazolyl, imidazolyl, oxadiazolyl, triazolyl, tetrazolyl, pyrimidinyl, pyrazinyl, indolyl, indolinyl and benzothiazolyl which are optionally substituted; heterocyclyl groups comprising pyrrolidinyl, thiazolidinyl, oxazolidinyl, morpholinyl, thiomorpholinyl, piperidinyl and piperazinyl which are optionally substituted, COR₁₆, wherein R₁₆ represents H; substituted or unsubstituted, (C₁-C₄) alkyl groups comprising methyl, ethyl, propyl, isopropyl, n-butyl, isobutyl and t-butyl; substituted or unsubstituted linear or branched, (C₂-C₇) alkenyl groups comprising ethenyl, propenyl and butenyl; aryl groups comprising phenyl and naphthyl, the aryl group is optionally substituted; aryloxy groups comprising phenoxy and napthoxy; substituted or unsubstituted linear or branched, (C₂-C₂₀) alkoxy groups comprising methoxy, ethoxy, propoxy, n-butoxy, isobutoxy and t-butoxy; suitable groups represented by D include CH₂ or C=O;
suitable substitutents are selected from halogens comprising fluorine, chlorine, bromine or iodine; hydroxy, nitro, cyano, azido, nitroso, amino, hydrazine, hydrazide, hydroxamate, formyl, alkyl, haloalkyl, haloalkoxy, cycloalkyl, aryl, alkoxy, aryloxy, acyl, acyloxy, acyloxyacyl, heterocyclyl, heteroaryl, monoalkylamino, dialkylamino, acylamino, alkylaminocarbonyl, alkoxycarbonyl, aryloxycarbonyl, heterocyclylcarbonyl, arylsulfonyl, alkylsulfinyl, arylsulfinyl, alkylthio, arylthio, sulfamoyl, alkoxyalkyl groups and carboxylic acids or its derivatives comprising carboxamide and carboxamidoalkyl.

10. A method of inhibiting the MAO enzyme for the treatment of a mental disorder according to claim 9, wherein the compound of formula (I) is selected from the group consisting of:
Methyl 2-amino-3-(4-{4-[(4-oxo-2-thioxo-1,3-thiazolidin-5-yl)methyl]phenoxy}phenyl)propanoate hydrochloride;
Methyl (2S)-2-amino-3-(4-{4-[(4-oxo-2-thioxo-1,3-thiazolidin-5-yl)methyl]phenoxy}phenyl)propanoate hydrochloride;
Methyl (2S)-2-amino-3-(4-{4-[(2,4-dioxo-1,3-thiazolidin-5-yl)methyl]-2- fluorophenoxy}phenyl)propanoate hydrochloride;
3-(2-Chloro-4-{4-[(3-methyl-2-oxo-1,3-oxazolidin-4-yl)methyl]phenoxy benzylidene)-1,3-dihydro-2H-indol-2-one;
3-(2-Chloro-4-{4-[(3-ethyl-2-oxo- 1,3-oxazolidin-4-yl) methyl]phenoxy}benzylidene)- 1,3-dihydro-2H-indol-2-one;
3-(2-Chloro-4-{4-[(5-oxomorpholin-3-yl)methyl]phenoxy}benzylidene) -1,3-dihydro-2H-indol-2-one;
Methyl (2S)-2-amino-3-(4-{3-chloro-4-[(2-oxo-1,2-dihydro-3H-indol-3 -ylidene) methyl]phenoxy}phenyl)propanoate hydrochloride;
Methyl (2S)-2-amino-3-(4-{2-chloro-4-[(2,4-dioxo-1,3 -thiazolidin-5- yl)methyl]- phenoxy}phenyl)propanoate hydrochloride;
[4-oxo-5 -(4-{4-[(3-ethyl-2-oxo- 1,3-oxazolidin-4-yl)methyl]phenoxy}benzyl)- 2-thioxo-1,3-thiazolidin-3 -yl]acetic acid;
4-(4-{2-chloro-4-[(4-oxo-2-thioxo-1,3-thiazolidin-5-yl)methyl]phenoxy}benzyl)-3-methyl-1,3-oxazolidin-2-one and
Methyl(2S)-2-amino-3-(4-{4-[(2,4-dioxo-1,3-thiazolidin-5- yl)methyl]phenoxy}phenyl)propanoate hydrochloride.

11. A method of inhibiting the MAO enzyme for the treatment of a mental disorder according to claim 9, wherein the mental disorder is selected from the group consisting of depression, anxiety, obsessional neuroses and atypical depression.

12. A method of inhibiting the MAO enzyme for the treatment of a mental disorder according to claim 9, wherein the mental disorder is depression.

13. A method for treating a disease, condition or disorder modulated by a cannabinoid receptor antagonist in animals comprising the step of administering to an animal in need of such treatment a therapeutically effective amount of a compound of formula (I), 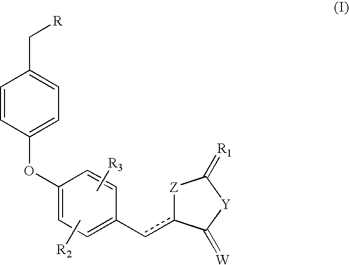
its derivatives, analogs, tautomeric forms, stereoisomers, polymorphs, hydrates, solvates, pharmaceutically acceptable salts, metabolites and prodrugs thereof, wherein, [symbol] represents optional bond;
W represents O or S;
Y represents NR₄, S or O, wherein R₄ represents hydrogen, substituted or unsubstituted groups selected from linear or branched (C₁-C₆) alkyl groups comprising methyl, ethyl, propyl, isopropyl, n-butyl, isobutyl and t-butyl; alkenyl groups comprising ethenyl, propenyl and butenyl; aryl groups comprising phenyl and naphthyl, the aryl group is optionally substituted; a counter ion is selected from alkali metals like Li, Na, and K; or —CH₂COOR₆, wherein R₆ represents H or an alkyl group;
Z represents CR₅ or S;
R₁ is selected from O, S or together with R₅ forms a fused 5 or 6 membered aromatic or heteroaromatic ring system containing carbon atoms or 1 or 2 hetereoatoms selected form O, S or N, comprising phenyl, naphthyl, furyl, pyrrolyl and pyridyl;
suitable groups represented by R₂, R₃ and R₅, are selected from hydrogen, halogens comprising fluorine, chlorine, bromine or iodine; hydroxy, nitro, cyano, formyl, amino, substituted or unsubstituted linear or branched (C₁-C₄) alkyl groups comprising methyl, ethyl, propyl, isopropyl, n-butyl, isobutyl and t-butyl; haloalkyl groups selected from alkyl groups substituted by one, two, three or four halogen atoms comprising chloromethyl, chloroethyl, trifluoromethyl, trifluoroethyl, dichloromethyl and dichloroethyl; substituted or unsubstituted, (C₁-C₄) alkoxy groups comprising methoxy, ethoxy, propoxy and butoxy;
R represents either A, B or C, wherein A represents 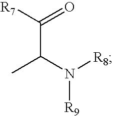
suitable groups represented by R₇ is selected from —OR₁₂, NR₁₃R₁₄; suitable groups represented by R₁₂ is selected from hydrogen, substituted or unsubstituted, linear or branched, (C₁-C₄) alkyl groups comprising methyl, ethyl, n-propyl, isopropyl, n-butyl, isobutyl and t-butyl; substituted or unsubstituted linear or branched, (C₂-C₄) alkenyl groups comprising ethenyl, propenyl and butenyl; aryl groups comprising phenyl and naphthyl, the aryl group is optionally substituted; aralkyl groups comprising benzyl, phenylethyl and phenylpropyl which are optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl and quinolinyl which are optionally substituted; the counter ion is selected from alkali metals like Li, Na, and K; alkaline earth metals like Ca and Mg; salts of different bases comprising ammonium or substituted ammonium salts, diethanolamine, α-phenylethylamine, benzylamine, piperidine, morpholine, pyridine, hydroxyethylpyrrolidine, hydroxyethylpiperidine, choline, aluminum and tromethamine;
suitable groups represented by R₁₃ and R₁₄ are selected from hydrogen, substituted or unsubstituted linear or branched, (C₁-C₄) alkyl groups comprising methyl, ethyl, n-propyl, isopropyl, n-butyl, isobutyl and t-butyl; substituted or unsubstituted linear or branched, (C₂-C₂₀) alkenyl groups comprising ethenyl, propenyl and butenyl; aryl groups comprising phenyl and naphthyl, the aryl group is optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl and quinolinyl;
suitable groups represented by R₈ and R₉ are selected from H, COR₁₅, substituted or unsubstituted linear or branched, (C₁-C₄) alkyl groups comprising methyl, ethyl, n-propyl, isopropyl, n-butyl, isobutyl and t-butyl; substituted or unsubstituted linear or branched, (C₂-C₄) alkenyl groups comprising ethenyl, propenyl and butenyl; aryl groups comprising phenyl and naphthyl, the aryl group is optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl and pyridazinyl which are optionally substituted;
suitable groups represented by R₁₅ are selected from hydrogen, substituted or unsubstituted linear or branched, (C₁-C₄) alkyl groups comprising methyl, ethyl, n-propyl, isopropyl, n-butyl, isobutyl and t-butyl; substituted or unsubstituted linear or branched, (C₂-C₄) alkenyl groups comprising ethenyl, propenyl and butenyl; aryl groups comprising phenyl and naphthyl, the aryl group is optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl, benzodioxolyl and quinolinyl; alkoxy groups comprising methoxy, ethoxy, n-propoxy and isopropoxy, which are optionally substituted; aralkoxy groups comprising phenylmethoxy, phenylethoxy and phenylpropoxy; aryloxy groups comprising phenoxy and napthoxy;
B represents 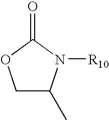
and C represents 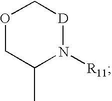
suitable groups represented by R₁₀ and R₁₁ are selected from H, alkyl, alkenyl, substituted or unsubstituted, (C₁-C₄) alkyl groups comprising methyl, ethyl, propyl, isopropyl, n-butyl, isobutyl and t-butyl; substituted or unsubstituted linear or branched, (C₂-C₇) alkenyl groups comprising ethenyl, propenyl and butenyl; aryl groups comprising phenyl and naphthyl, the aryl group is optionally substituted; aryloxy groups comprising phenoxy and napthoxy substituted or unsubstituted linear or branched, (C₂-C₄) alkoxy groups comprising methoxy, ethoxy, propoxy and n-butoxy; heteroaryl groups comprising pyridyl, thienyl, furyl, pyrrolyl, oxazolyl, thiazolyl, imidazolyl, oxadiazolyl, triazolyl, tetrazolyl, pyrimidinyl, pyrazinyl, indolyl, indolinyl and benzothiazolyl which are optionally substituted; heterocyclyl groups comprising pyrrolidinyl, thiazolidinyl, oxazolidinyl, morpholinyl, thiomorpholinyl, piperidinyl and piperazinyl which are optionally substituted, COR₁₆, wherein R₁₆ represents H; substituted or unsubstituted, (C₁-C₄) alkyl groups comprising methyl, ethyl, propyl, isopropyl, n-butyl, isobutyl and t-butyl; substituted or unsubstituted linear or branched, (C₂-C₇) alkenyl groups comprising ethenyl, propenyl and butenyl; aryl groups comprising phenyl and naphthyl, the aryl group is optionally substituted; aryloxy groups comprising phenoxy and napthoxy; substituted or unsubstituted linear or branched, (C₂-C₂₀) alkoxy groups comprising methoxy, ethoxy, propoxy, n-butoxy, isobutoxy and t-butoxy; suitable groups represented by D include CH₂ or C=O;
suitable substitutents are selected from halogens comprising fluorine, chlorine, bromine or iodine; hydroxy, nitro, cyano, azido, nitroso, amino, hydrazine, hydrazide, hydroxamate, formyl, alkyl, haloalkyl, haloalkoxy, cycloalkyl, aryl, alkoxy, aryloxy, acyl, acyloxy, acyloxyacyl, heterocyclyl, heteroaryl, monoalkylamino, dialkylamino, acylamino, alkylaminocarbonyl, alkoxycarbonyl, aryloxycarbonyl, heterocyclylcarbonyl, arylsulfonyl, alkylsulfinyl, arylsulfinyl, alkylthio, arylthio, sulfamoyl, alkoxyalkyl groups and carboxylic acids or its derivatives comprising carboxamide and carboxamidoalkyl.

14. A method for treating a disease, condition or disorder modulated by a cannabinoid receptor antagonist in animals according to claim 13, wherein said disease, condition or disorder modulated by a cannabinoid receptor antagonist is selected from the group consisting of weight loss, obesity, bulimia, depression, bipolar disorders, psychoses, schizophrenia, behaviors, alcoholism, tobacco abuse, memory loss, Alzheimer's disease, dementia of aging, seizure disorders, epilepsy, attention deficit disorder, and Parkinson's disease.
